(19) United States
(12) Patent Application Publication
(10) Pub. No.: US 2024/0175868 A1
(43) Pub. Date: May 30, 2024

(54) Enzyme-linked paper-based assay

(71) Applicant: Anna Toldra Filella, Danderyd (SE)

(72) Inventors: Anna Toldra Filella, Danderyd (SE); Mahiar Max Hamedi

(21) Appl. No.: 18/518,704

(22) Filed: Nov. 24, 2023

Related U.S. Application Data
(60) Provisional application No. 63/546,556, filed on Oct. 31, 2023.

(30) Foreign Application Priority Data
Nov. 29, 2022 (SE) SE-2230391-1

(51) Int. Cl. G01N 33/543; B01L 3/00

(52) U.S. Cl. CPC G01N 33/54391; B01L 3/5023; B01L 2200/16; B01L 2300/069

(57) ABSTRACT

A vertical flow microfluidic device configured and a method for performing enzyme-linked assays are disclosed. The device comprises several layers of porous materials, termed “microfluidic enzyme-linked paper analytical device (μEL-PAD)”. The device exploits a one-step sandwich coupling to form beads/analyte/enzyme complexes, which are subsequently added to a vertical flow paper wherein a nitrocellulose membrane retains them. This ability to entrap the bead complexes without disrupting the flow allows for an easy washing step using absorbent/barrier layers that are manually pulled out. Finally, the chromogenic substrate stored on the detection paper reacts with at least the enzyme-linked complexes, generating a blue color on the paper that is quantified with a smartphone app after removal of the nitrocellulose layer. The device can detect many analytes, for example, DNA with an enzyme-linked oligonucleotide assay (ELONA) or antigen (e.g. protein) with enzyme-linked immunosorbent assay (ELISA).

### CROSS-REFERENCE TO RELATED APPLICATIONS

This application claims the benefit of Swedish application No. 2230391-1, filed on Nov. 29, 2022, and claims priority to U.S. provisional application Ser. No. 63/546,556, filed Oct. 31, 2023, the entire contents of which are hereby incorporated by reference.

### BACKGROUND OF THE INVENTION

Enzyme-linked assays, like enzyme-linked immunosorbent assays (ELISAs), are the standard methods for colorimetric biomarker detection since they offer high sensitivity due to an enzymatic signal amplification. Enzyme-linked assays offer excellent precision and high throughput analysis as they are commonly realized using microtiter plates. However, such assays require expensive, bulky equipment (i.e., microplate readers) for signal quantification, expertise of operation, and laborious protocols involving series of mixing, incubation and washing steps. Therefore, they remain laboratory-based approaches that are not well suited for point-of-care (POC) testing.

POC devices that enable timely results at the point of need are fundamental in healthcare and medicine, or in many other fields like environmental monitoring, food safety, and security. In this sense, lateral flow assays (LFAs) for POC have existed for decades. The initial big area of use for LFAs was over-the-counter home pregnancy tests. Most recently, their massive proliferation for SARS-CoV-2 antigen detection has played a crucial role during the pandemic. LFAs bypass the laborious and manual steps required in enzyme-linked assays by exploiting the capillary properties of paper to move fluids, making them easy to use and well-suited for on-site detection in resource-limited settings. Other appealing properties of paper-based devices include the capability of storing reagents into the cellulose matrix to have ready-to-use substrates, cost effectiveness, mass manufacturing, disposability, and the possibility to incinerate paper.

Despite the listed advantages, traditional LFAs inherently have inferior analytical performance than enzyme-linked assays, as they exploit gold nanoparticle labels that cannot amplify the colorimetric signal. Another reason of the poor sensitivity in LFAs is the limited time for the biorecognition event to take place.[1] Moreover, they are also qualitative, relying on a subjective visual readout based on the presence or absence of the analyte.[2-4] To tackle the latter, smartphone-assisted devices that allow for acquisition and quantification of the signal by a smartphone, while avoiding expensive instruments and misinterpretation of results, are a good alternative.[5, 6]

To increase the specificity and sensitivity of LFAs, some attempts have been carried out to develop enzyme-linked assays on paper analytical devices. The first paper-based ELISA was demonstrated by Whitesides' group in 2010,[7] and other works followed.[9-11] However, these demonstrations require multiple steps that are carried out outside the paper, not being microfluidic in nature. Although microfluidic ELISAs have been reported combining paper and fluidic polyester channels,[3, 12] works that describe ELISA in a microfluidic paper analytical device (“μPAD”) are very few.[4, 13-15] These works demonstrated notable progress in the field, but they conducted insufficient washing steps,[13, 15] required multiple timed steps,[4, 16] lacked sufficient sensitivity and accuracy,[4] and/or did not undergo comparison with currently used technologies in terms of analytical characteristics,[14] besides none of them being quantitative. Overall, these factors limit their practicality and deployment for POC testing in real-world situations.

Therefore, a need remains to incorporate enzyme-linked assays into μPADs to develop easy-to-use POC tests with high analytical performance.

The present application relates to the use of paper-based bioanalytical devices “microfluidic enzyme-linked paper analytical device (μEL-PAD)” that are designed to carry out enzyme-linked assays.

### SUMMARY OF THE INVENTION

In a first aspect of the invention there is disclosed a vertical flow microfluidic device configured for performing chromogenic assays, comprising: an inlet layer for delivering a solution comprising enzyme-linked complexes, a membrane layer comprising a porous membrane for retaining, immobilizing or adsorbing the enzyme-linked complexes, at least one absorbent/barrier layer for absorbing the solution comprising enzyme-linked biorecognition molecule conjugates, and a detection layer configured for comprising a chromogenic substrate and changing color when the enzyme-linked complexes react with the chromogenic substrate.

In one embodiment of the vertical flow microfluidic device, the solution comprising enzyme-linked complexes further comprises a plurality of: an analyte/primary (anti-analyte) enzyme-linked biorecognition molecule conjugate; an analyte/primary (anti-analyte) biorecognition molecule/secondary (anti-primary biorecognition molecule) enzyme-linked biorecognition molecule conjugate; an analyte immobilized on a support/primary (anti-analyte) enzyme-linked biorecognition molecule conjugate; an analyte immobilized on a support/primary (anti-analyte) biorecognition molecule/secondary (anti-primary biorecognition molecule) enzyme-linked biorecognition molecule conjugate; a biorecognition molecule/analyte/primary (anti-analyte) enzyme-linked biorecognition molecule conjugate; a biorecognition molecule/analyte/primary (anti-analyte) biorecognition molecule/secondary (anti-primary biorecognition molecule) enzyme-linked biorecognition molecule conjugate; a biorecognition molecule immobilized on a support/analyte/primary (anti-analyte) enzyme-linked biorecognition molecule conjugate; or a biorecognition molecule immobilized on a support/analyte/primary (anti-analyte) biorecognition molecule/secondary (anti-primary biorecognition molecule) enzyme-linked biorecognition molecule conjugate.

In one embodiment of the vertical flow microfluidic device, the solution comprising enzyme-linked complexes further comprises a buffer, chromogenic substrate, or oxidizing agent for the enzyme of the enzyme-linked biorecognition molecule conjugates.

In one embodiment of the vertical flow microfluidic device, the analyte comprises cells, viruses, virus particles, antigens, antibodies, proteins, protein fragments, hormones, peptides, DNA, RNA, toxins, small molecules, metabolites and/or drugs.

In one embodiment of the vertical flow microfluidic device, wherein the enzyme of the enzyme-linked biorecognition molecule conjugate comprises HRP, ALP, or β-galactosidase.

In one embodiment of the vertical flow microfluidic device, the biorecognition molecule comprises DNA fragments, RNA fragments, antibodies, antibodies fragments, or engineered molecules.

In one embodiment of the vertical flow microfluidic device, the immobilization support comprises particles, magnetic beads, and other materials.

In one embodiment of the vertical flow microfluidic device, the chromogenic substrate comprises 2,2′-Azinobis [3-ethylbenzothiazoline-6-sulfonic acid]-diammonium salt, ABTS (substrate for horseradish peroxidase, HRP), o-phenylenediamine dihydrochloride, OPD (substrate for HRP), 3,3′,5,5′-tetramethylbenzidine (substrate for HRP), p-Nitrophenyl Phosphate, PNPP (substrate for alkaline phosphatase, ALP), or o-nitrophenyl-β-D-galactopyranoside, ONPG (substrate for β-galactosidase).

In one embodiment of the vertical flow microfluidic device, the oxidizing agent is hydrogen peroxide.

In one embodiment of the vertical flow microfluidic device, the chromogenic substrate is configured for storage in dried and lyophilized forms.

In one embodiment of the vertical flow microfluidic device, the membrane layer comprises a nitrocellulose membrane.

In one embodiment of the vertical flow microfluidic device, the nitrocellulose membrane is configured for specifically or non-specifically adsorbing, retaining or immobilizing the enzyme-linked complexes.

In a second aspect of the invention there is provided a method of using the vertical flow microfluidic device comprising the steps of: delivering a solution comprising enzyme-linked complexes onto the inlet layer; adsorbing, retaining or immobilizing enzyme-linked complexes on the membrane layer; absorbing the solution containing enzyme-linked biorecognition molecule conjugates in the absorbent/barrier layer; optionally adding a washing buffer to the membrane layer; absorbing the washing buffer in the absorbent/barrier layer; removing the absorbent/barrier layer; contacting the complexes in membrane layer with the detection layer comprising the chromogenic substrate and incubating with a solution comprising buffer, chromogenic substrate, or oxidizing agent to react and cause a color change; optionally removing the membrane layer, and detecting the color change.

In one embodiment, the method comprises the step of before contacting the membrane layer with the detection layer, adding chromogenic substrates to the detection layer.

In one embodiment of the method the color change is detected on the membrane layer or the detection layer.

In one embodiment of the method, the step of detecting the color change further comprises quantifying the color change.

In one embodiment of the method, quantifying the color change comprises using a smartphone for quantifying the color change.

### Definitions

In this application, the term analyte refers to the substance to be detected. The analyte comprises cells, viruses, virus particles, antigens, antibodies, proteins, protein fragments, hormones, peptides, DNA, RNA, toxins, small molecules, metabolites and/or drugs.

In this application, the term biorecognition molecule refers to a molecule that specifically interacts with another molecule. The biorecognition molecule interacts with molecules comprising the analyte ((primary (anti-analyte) biorecognition molecule) or other biorecognition molecules ((secondary (anti-primary) biorecognition molecule)). The biorecognition molecule comprises DNA fragments, RNA fragments, antibodies, antibodies fragments, or engineered molecules.

In this application, the term chromogenic substrate refers to a molecule that produces the color change as a result of an enzymatic reaction. The chromogenic substrate can directly react with the enzyme, or it can require an oxidizing agent like hydrogen peroxide for the enzymatic reaction to take place. The chromogenic substrate comprises 2,2′-Azinobis [3-ethylbenzothiazoline-6-sulfonic acid]-diammonium salt, ABTS (substrate for horseradish peroxidase, HRP), o-phenylenediamine dihydrochloride, OPD (substrate for HRP), 3,3′,5,5′-tetramethylbenzidine (substrate for HRP), p-Nitrophenyl Phosphate, PNPP (substrate for alkaline phosphatase, ALP), or o-nitrophenyl-β-D-galactopyranoside, ONPG (substrate for β-galactosidase).

In this application, the term enzyme-linked complex refers to a complex generated through specific interaction between molecules that further includes an enzyme-linked conjugate. Enzyme-linked complexes can comprise:
an analyte/primary (anti-analyte) enzyme-linked biorecognition molecule conjugate;
an analyte/primary (anti-analyte) biorecognition molecule/secondary (anti-primary biorecognition molecule) enzyme-linked biorecognition molecule conjugate;
an analyte immobilized on a support/primary (anti-analyte) enzyme-linked biorecognition molecule conjugate;
an analyte immobilized on a support/primary (anti-analyte) biorecognition molecule/secondary (anti-primary biorecognition molecule) enzyme-linked biorecognition molecule conjugate;
a biorecognition molecule/analyte/primary (anti-analyte) enzyme-linked biorecognition molecule conjugate;
a biorecognition molecule/analyte/primary (anti-analyte) biorecognition molecule/secondary (anti-primary biorecognition molecule) enzyme-linked biorecognition molecule conjugate;
a biorecognition molecule immobilized on a support/analyte/primary (anti-analyte) enzyme-linked biorecognition molecule conjugate;
a biorecognition molecule immobilized on a support/analyte/primary (anti-analyte) biorecognition molecule/secondary (anti-primary biorecognition molecule) enzyme-linked biorecognition molecule conjugate.

In this application, the term immobilization support refers to the material that captures molecules comprising the analyte, biorecognition molecule or enzyme-linked complexes. The immobilization support comprises particles, magnetic beads, and other materials.

It must be noted that as used herein, the singular forms “a”, “an”, and “the”, include plural references unless the context clearly indicates otherwise. Thus, for example, reference to “molecule” includes one or more of the molecules disclosed herein and reference to “the method” includes reference to equivalent steps and methods known to those of ordinary skill in the art that could be modified or substituted for the methods described herein. The same applies to other terms such as complex, enzyme, analyte, substrate, etc.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integer or step. When used herein the term “comprising” can be substituted with the term “containing” or sometimes when used herein with the term “having”.

When used herein “consisting of” excludes any element, step, or ingredient not specified in the claim element. When used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms.

The term “about” or “approximately” as used herein means within 20%, preferably within 10%, and more preferably within 5% of a given value or range. It includes also the concrete number, e.g., about 20 includes 20.

Unless otherwise defined herein, scientific and technical terms used in connection with the present invention shall have the meanings that are commonly understood by those of ordinary skill in the art. Further, unless otherwise required by context, singular terms shall include pluralities and plural terms shall include the singular. The methods and techniques of the present invention are generally performed according to conventional methods well-known in the art. Generally, nomenclatures used in connection with techniques of biochemistry, enzymology, molecular and cellular biology, microbiology, genetics and protein and nucleic acid chemistry and hybridization described herein are those well-known and commonly used in the art.

### BRIEF DESCRIPTION OF DRAWINGS

FIG. 1 Shows the components and fabrication process of the microfluidic enzyme-linked paper analytical device (μEL-PAD): 1) Different porous materials were printed, melted and/or cut; 2) The layers were assembled; 3) 3,3′,5,5′-tetramethylbenzidine (TMB) was stored in a dried form on the detection layer; and 4) The device was assembled.

FIGS. 2A-2C show the schematic procedure of the μEL-PAD for DNA detection, including: FIG. 2A—Recombinase polymerase amplification (RPA) to amplify DNA and generate RPA products flanked with biotin and fluorescein (FITC); FIG. 2B—One-step sandwich coupling to obtain beads/RPA products/antiFITC-horseradish peroxidase (HRP) complexes; FIG. 2C—Paper microfluidic device to conduct washing and detection steps: i) Addition of the bead complexes, which were retained on the membrane layer; ii) Addition of the washing buffer. After complete absorption of the buffer, the intermediate layers were manually removed; iii) Addition of hydrogen peroxide (H2O2) and 10 min incubation to generate the blue color by the enzymatic reaction; iv) Color readout after removal of the membrane layer.

FIGS. 3A-3B show the results of the μEL-PAD and comparison with the results obtained using a lateral flow assay (LFA): FIG. 3A shows the calibration curves and photos of the μEL-PAD; and FIG. 3B shows the calibration curves and photos of the LFA. The dotted horizontal lines represent the LODs, calculated as the mean of the corresponding blank (NTC=no template control) values plus 3 times their SD. The LOD of the μEL-PAD is lower than 10000 DNA target copies/μL. The LOD of the LFA is 700 DNA target copies/μL.

FIG. 4 shows the results of the one-step sandwich coupling in solution using different volume of beads. After washing, H2O2 and TMB were added. Absorbance was read at λ=620 nm. Positive samples contain 104 DNA target copies/μL; Blanks (NTC=no template control) contain nuclease-free water.

FIG. 5. shows the calibration curve and photos obtained using serially diluted antiFITC-HRP and 100 μL of TMB stored on filter paper. Results were obtained after 10 min incubation with the antiFITC-HRP and H2O2. A smartphone app. was used for color quantification from the photos. The dotted horizontal line represents the LOD, calculated as the mean of the corresponding blank (NTC=no template control) values plus 3 times their SD. The LOD is lower than 5 antiFITC-HRP mU.

FIGS. 6A-6B show the schematic procedure of the μEL-PAD for hormone detection, including: FIG. 6A shows one-step coupling to obtain hormone/anti-hormone antibody-HRP conjugate complexes. FIG. 6B shows the vertical flow paper microfluidic device to conducting washing and detection steps: i) Addition of the enzyme-linked complexes, which are specifically immobilized on the membrane layer; ii) Addition of the washing buffer. After complete absorption of the buffer, the absorbent/barrier layer is manually removed; iii) Addition of buffer, incubation to generate the blue color by the enzymatic reaction and color readout. The hydrogen peroxide (H2O2) and TMB are stored on the detection layer.

### DETAILED DESCRIPTION OF THE INVENTION

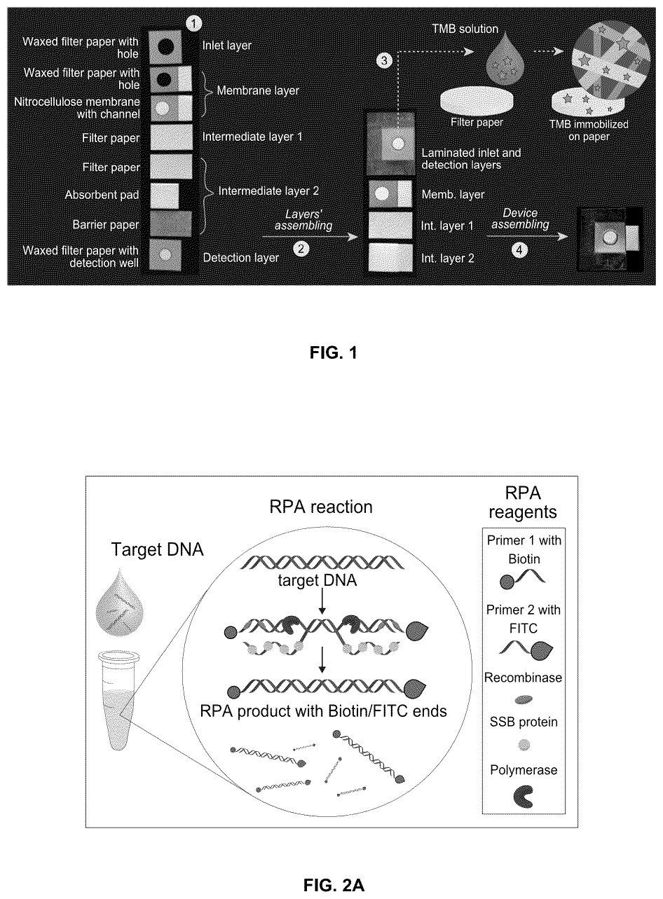

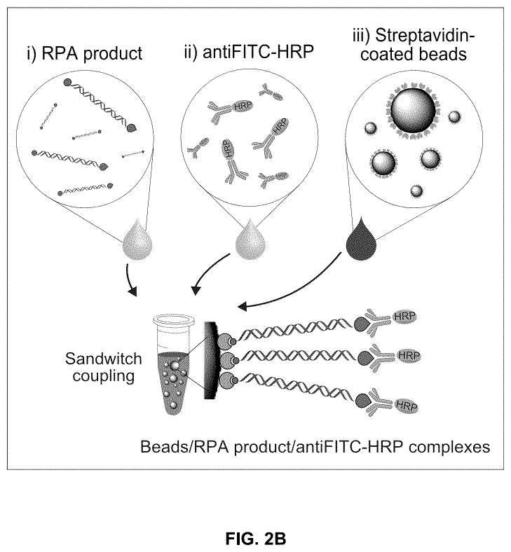

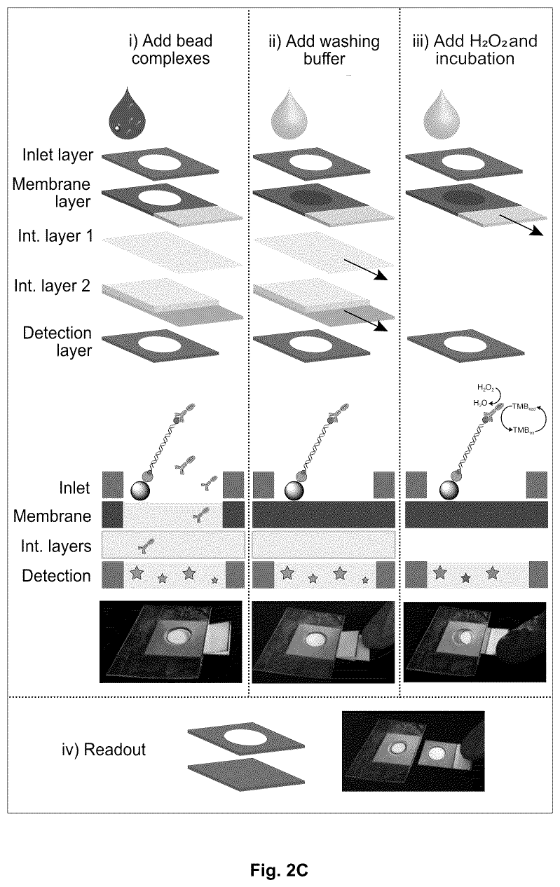

In an embodiment, a “microfluidic enzyme-linked paper analytical device (μEL-PAD)” is fabricated using different materials including a Whatman Grade 1 filter paper, nitrocellulose membrane of 3 μm in pore size, a Whatman CF6 cotton absorbent pad and a Whatman weighing paper Grade B-2 Kjeldahl. These materials are used to fabricate 5 layers composing the 3D vertical device as seen in FIG. 1: 1) Inlet layer: waxed filter paper with a hole (1.1 cm diameter); 2) Movable membrane layer: waxed nitrocellulose membrane with hydrophilic channel (1 cm diameter) attached to a waxed filter paper with a hole (1 cm diameter); 3) Movable 1st intermediate layer: filter paper; 4) Movable 2nd intermediate layer: composed of a filter paper, absorbent pad and weighing paper; 5) Detection layer: waxed filter paper with a hydrophilic well where 3,3′,5,5′-tetramethylbenzidine (TMB) was stored. Each of these layers contribute to a specific function in the microfluidic device as seen in FIG. 2C: 1) Inlet layer: it allows introduction of the sample; 2) Movable membrane layer: it retains the bead complexes while allowing the supernatant to pass through the membrane; 3) Movable 1st intermediate layer: it adsorbs the supernatant and washing buffer: 4) Movable 2nd intermediate layer: it absorbs the washing buffer while acting as a barrier; 5) Detection layer: it contains the stored TMB, interacts with the washed bead complexes on the nitrocellulose membrane, and turns blue after addition and incubation of hydrogen peroxide (H2O2).

In one alternative embodiment of the device above, a design can have a hole/channels/wells in the layers with decreasing diameter sizes for each hole: the inlet layer having a hole of 1.1 cm diameter, the movable membrane layer having a channel with 1 cm diameter, and the detection layer having a well of 0.9 cm diameter. These channel patterns can be printed into the paper with a hydrophobic material, a most viable option being wax printing to print wax pattern, and subsequently the wax melted into the paper using heat such as a hot press. Wax print of nitrocellulose membranes can also be done using transfer of wax printed patterns from other papers such as commercial print paper, by hot pressing the two sheets together at above 100° C.

In one alternative embodiment, the nitrocellulose membrane has a pore size smaller than the beads so that the beads are retained in the nitrocellulose and do not pass through it. The nitrocellulose membrane is further contact with a filter paper underneath to allow best flow profile.

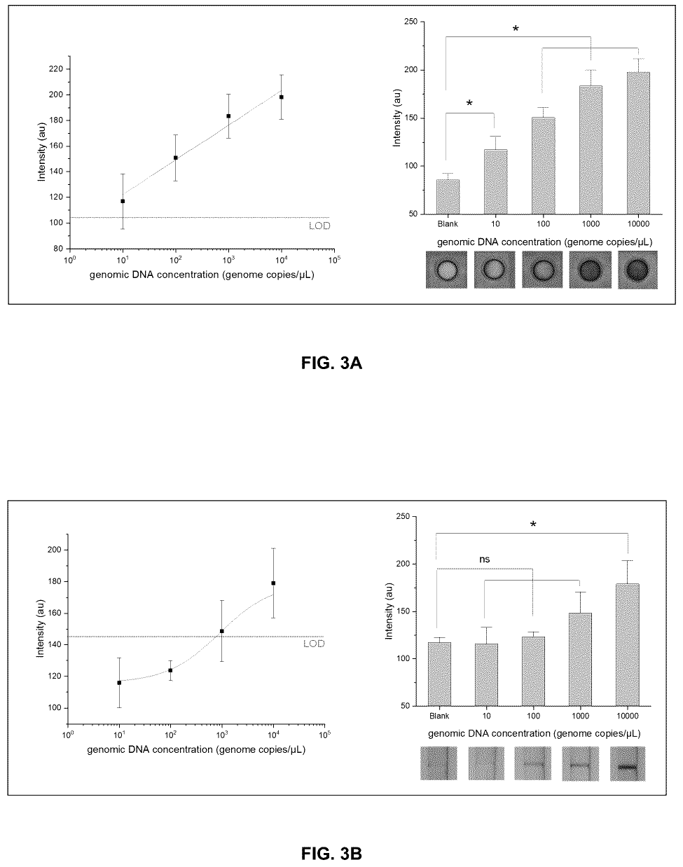

In one alternative embodiment, the μEL-PAD device performs an enzyme-linked oligonucleotide assay (ELONA). This assay requires a DNA amplification step. To bypass the thermal cycling and be therefore more amenable for point of care (POC) testing, isothermal amplification techniques such as Recombinase polymerase amplification (RPA), which operates at low temperatures of 37 to 42° C., can be used. To perform the assay, Biotin-labelled primers and fluorescein (FITC)-labelled primers were used to generate an RPA product with Biotin and FITC at each end, as seen in FIG. 2A. The enzyme-linked assay comprises a first one-step sandwich coupling, followed by washing and detection steps on the paper device. The sandwich coupling can be performed in suspension exploiting streptavidin-coated magnetic beads and antiFTIC-horseradish peroxidase (HRP) conjugates, as seen in FIG. 2B. The mixture is then added into the μEL-PAD, in which the following steps happen, as seen in FIG. 2C: 1) The bead complexes are retained on the nitrocellulose membrane; 2) The intermediate adsorbent pad layers absorb the supernatant and washing buffer (both containing excess of enzyme conjugate) while also stopping the flow; and 3) A detection layer where TMB is stored by first adding solutions of TMB to the paper and drying the solution for storage. This layer is brought to contact with the nitrocellulose layer after washing steps. Finally, a solution of H2O2 is added into the device and the color change quantified with the smartphone app after 10 min incubation and removal of the membrane layer. The analytical performance of this systems is calculated and compared with that obtained with a conventional LFA, as seen in FIG. 3. The μEL-PAD showed an improvement of the LOD of at least 70 times compared to the LFA.

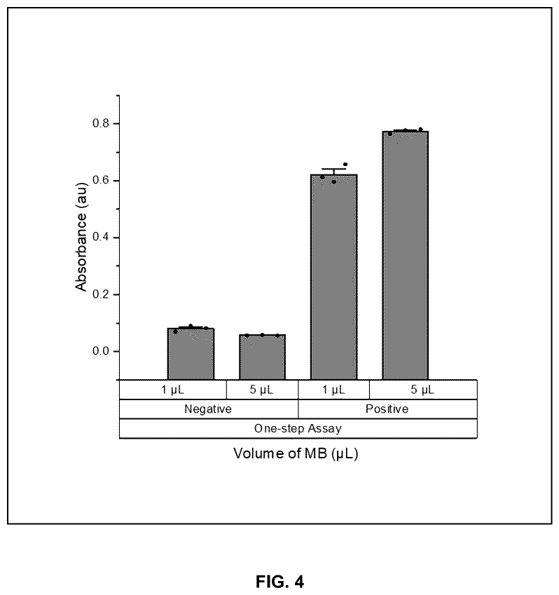

In one alternative embodiment of the said one-step sandwich coupling in suspension, the following optimized conditions can be used: 1) One-step assay in which non-diluted RPA product and antiFITC-HRP in Casein-phosphate buffered saline (PBS) are mixed and incubated for 30 min under shaking, following incubation with streptavidin-coated magnetic beads (concentration of 10 mg/mL with diameter size of 1 μm) for 30 min under shaking. The one-step assay using non-diluted RPA product can discriminate between blank and a positive sample like a standard three-step assay using 1:100 diluted RPA; 2) Blocking with Casein-PBS, as it shows lower non-specific values than bovine serum albumin (BSA)-PBS; 3) The one-step assay can use 1 and 5 μL of magnetic beads. The positive signals increase with increasing amounts of beads and the non-specific signals decrease with increasing amounts of beads as seen in FIG. 4. Therefore, 5 μL can be an optimal solution for this ELONA and for integration into paper.

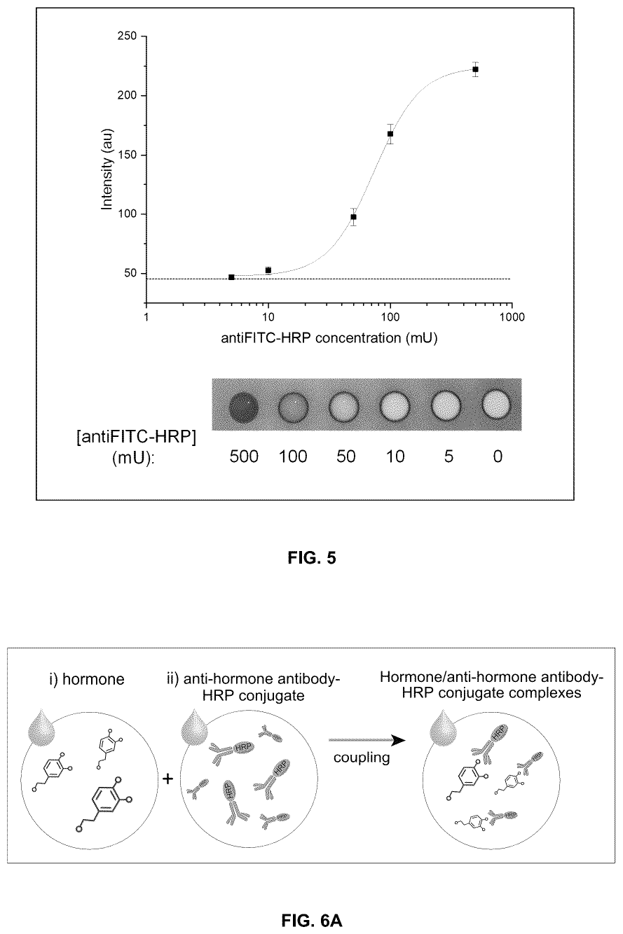

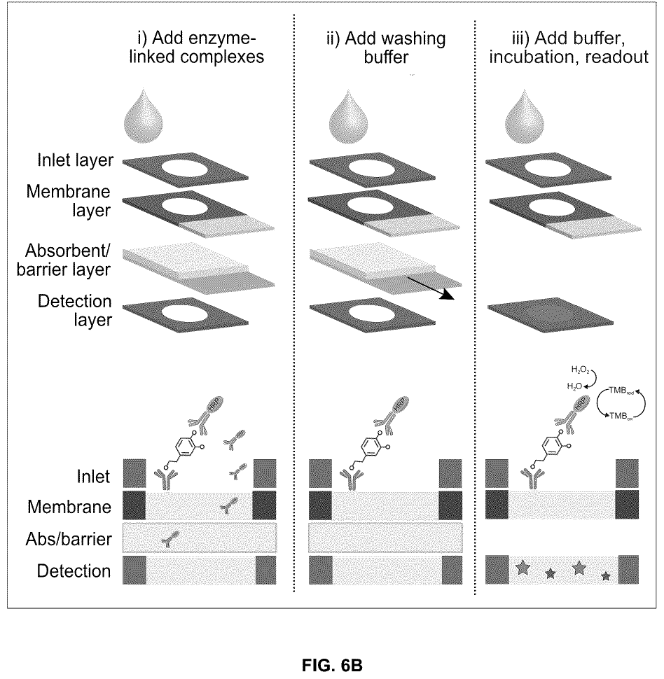

For the detection on paper, different volumes of TMB (e.g. 50 μL, 100 μL or 200 μL at 0.4 g/L) can be dropped on paper and dried it overnight. After adding 5 μL of serially diluted antiFITC-HRP with 45 μL 0.02% H2O2, 10 min incubation and a smartphone readout, the LOD obtained for 50 μL of dried TMB on paper was like that obtained for 50 μL TMB in solution, with LODs higher than 10 mU of antiFITC-HRP. When using 100 μL or 200 μL of TMB stored on paper and reading the output color after 10 min, the LOD was reduced to lower than 5 mU of antiFITC-HRP. As seen in FIG. 5, 100 μL of dried TMB can be optimal for storing TMB on paper to conduct this microfluidic enzyme-linked assay on paper.

In one embodiment, the colorimetric readout of the assay can be done with human eyes. In another mode, a smartphone camera can be used to take a photo and further digital analysis using a software to get digital color information. Paper provides the added advantage of being less affected by ambient light conditions and had much lower optical aberrations than plastic tubes or microplates, which are commonly used today for performing enzyme linked assays, as plates reflect the ambient light at some angels leading to wrong quantitative values of the signal.

In one embodiment, the μEL-PAD can also be used for detection of many other analytes besides DNA, such as proteins with an enzyme-linked immunosorbent assay (ELISA), or other formats besides sandwich, such as direct, indirect, competitive, or other strategies based on the use of enzyme-linked molecules.

An analytical device comprises a vertical flow paper microfluidic system composed of at least two porous substrates in contact, where enzyme-linked complexes are retained on the first substrate. A solution containing a chromogenic substrate flows from the first to the second porous substrate, resulting in an enzymatic reaction that leads to a color change of the chromogenic substrate and the porous substrates.

Said device according including an absorbent/barrier layer between the two porous substrates. A solution containing enzyme-linked complexes is added to the device, these being retained on the first porous substrate. A washing buffer is subsequently added to remove the excess of enzyme-linked conjugates, which are absorbed by the absorbent layer. The barrier layer is removed, bringing the two porous substrates into contact.

Said device where the first porous substrate is removed after color generation, the color readout being performed on the second porous substrate.

Said device where the first porous substrate retains the enzyme-linked complexes by size. The first porous substrate including but not limited to nitrocellulose membranes having pores smaller than the enzyme-linked complexes' dimensions.

Said device containing the chromogenic substrate stored on the second porous substrate. The chromogenic substrate including but not limited to 2,2′-Azinobis [3-ethylbenzothiazoline-6-sulfonic acid]-diammonium salt, ABTS (substrate for horseradish peroxidase, HRP), o-phenylenediamine dihydrochloride, OPD (substrate for HRP), p-Nitrophenyl Phosphate, PNPP (substrate for alkaline phosphatase, ALP), o-nitrophenyl-β-D-galactopyranoside, ONPG (substrate for β-galactosidase), directly reacting with the enzyme or requiring an oxidizing agent (hydrogen peroxide).

The enzyme-linked complexes used in said device, may include but are not not limited to: 1) Cells, viruses, virus particles, antigens, antibodies, proteins, protein fragments, hormones, peptides, DNA, RNA, toxins, small molecules, metabolites and/or drugs, which are bound to an enzyme-linked conjugate, or 2) Cells, viruses, virus particles, antigens, antibodies, proteins, protein fragments, hormones, peptides, DNA, RNA, toxins, small molecules, metabolites and/or drugs, which are immobilized on a support (including but not limited to particles, magnetic beads, and other materials with a bigger size than the pore size of the first porous substrate in claim 1) and bound to an enzyme-linked conjugate. The enzyme-linked conjugates composing the enzyme-linked complexes including but not limited to HRP, ALP, or β-galactosidase.

The enzyme-linked complexes can be generated from a direct, indirect, sandwich, competitive, or other enzyme-linked assay formats. The enzyme-linked complexes including compositions of: 1) The analyte from the sample that binds directly to an enzyme-linked anti-analyte biorecognition molecule conjugate or indirectly using an intermediate anti-analyte unconjugated biorecognition molecule (direct and indirect formats); 2) A biorecognition molecule that binds to the analyte from the sample, following binding to an enzyme-linked anti-analyte biorecognition molecule conjugate, directly or indirectly (sandwich enzyme-linked format); 3) A synthetic analyte that binds to the enzyme-linked anti-analyte biorecognition molecule conjugate, directly or indirectly, this conjugate competing with the analyte in the sample (competitive format); 4) The enzyme-linked conjugate after interacting with the analyte (other format). The biorecognition molecule comprising anti-analyte biorecognition molecule or secondary anti-primary biorecognition molecule, which can be conjugated to an enzyme. The enzyme composing the enzyme-linked conjugates including but not limited to HRP, ALP, or β-galactosidase.

The analyte can include but is not limited to cells, viruses, virus particles, antigens, antibodies, proteins, protein fragments, hormones, peptides, DNA, RNA, toxins, small molecules, metabolites and/or drugs.

The sandwich enzyme-linked format to detect oligonucleotides comprising: streptavidin-coated magnetic beads that bind to nucleic acid fragments flanked with Biotin and fluorescein (FITC), and antiFITC-HRP conjugates. Resulting complexes are streptavidin-magnetic bead/nucleic acid fragment with Biotin and FITC/antiFITC-HRP conjugate. The nucleic acid fragments with Biotin and FITC are generated by Recombinase polymerase amplification (RPA).

Said device having 5 layers to conduct an enzyme-linked oligonucleotide assay (ELONA) with washing and detection steps exploiting porous materials: 1) Layer 1: waxed filter paper with a hole where enzyme-linked complexes in are introduced; 2) Layer 2: waxed nitrocellulose membrane with a hydrophilic channel where enzyme-linked complexes are retained by size; 3) Layer 3 and 4: filter paper, absorbent pad, and hydrophobic paper to remove excess of antiFITC-HRP conjugates present with the enzyme-linked complexes; 4) Layer 5: waxed filter paper with a hydrophilic well where TMB is stored. After addition of washing buffer to the inlet of the device, the layers 3 and 4 containing excess of antiFITC-HRP conjugates are pulled out, bringing layer 2 and layer 5 into contact. After addition of H2O2 to the inlet of the device, the solution flows to the layer 5. After incubation for the enzymatic reaction to take place, the layer 2 is removed and the color change of the chromogenic substrate on layer 5 is measured.

### Example—Manufacturing a Vertical Flow Microfluidic Device

In this embodiment a vertical flow microfluidic device with five layers was produced (FIG. 1C) comprising: (1) an inlet layer: waxed filter paper with a circle hole; (2) a membrane layer: waxed nitrocellulose membrane with hydrophilic channel attached to a waxed filter paper with a circle hole; (3) a 1st intermediate layer: filter paper; (4) a 2nd intermediate layer: layer composed of a filter paper, absorbent pad, and barrier paper (weighing paper); and (5) a detection layer: waxed filter paper with a hydrophilic well where TMB is stored.

The device design was created using AutoCAD (Autodesk Inc., USA). The patterns of the layers of the device were wax printed on Whatman Grade 1 filter paper using a wax printer (Xerox color Qube 8570/8870, Malaysia). The wax was subsequently melted using hot press (Skilte Produktion E-15S, China) at 140° C. for 1 min. The patterns were cut using a cutting machine (Brother ScanNCut CM900, China). Wax printed filter paper layers included: waxed filter paper with a circle hole (1.1 cm diameter); waxed filter paper with a circle hole (1 cm diameter); and waxed filter paper with a hydrophilic channel (0.9 cm diameter). Additionally, DinA4 office paper was wax printed with a hydrophilic circle (1 cm diameter). After adding the nitrocellulose membrane on the top of the wax printed office paper, the wax was melted at 120° C. for 1 min to transfer the pattern to the nitrocellulose membrane.

FIG. 1 shows the fabrication of the device in four steps: 1) Different porous materials were wax printed, melted at 140° C. for 1 min and/or cut. The inlet layer, made of waxed filter paper with hole, was wax printed, melted and cut. The membrane layer was composed of a waxed printed filter paper with a hole and a nitrocellulose membrane with a channel. The filter paper was wax printed, melted and cut. To fabricate the nitrocellulose membrane, office paper was wax printed and put in contact with the membrane. Subsequently, the wax was melted at 120° C. for 1 min to transfer the pattern to the nitrocellulose membrane. The intermediate layer 1, made of filter paper, was cut. The filter paper, absorbent pad and barrier paper composing the intermediate layer 2 were cut. The detection layer, made of waxed filter paper with a well, was printed, melted, and cut; 2) The porous substrates composing the membrane layer and intermediate layer 2 were aligned and attached with double-sided adhesive tape. The inlet and detection layers were aligned and attached using double-sided adhesive tape, following lamination between two plastic substrates at 140° C. for 1 min. One side of the laminated device was cut to enable subsequent insertion of the membrane and intermediate layers; 3) 50 μL of TMB were added to the hydrophilic well of the detection layer and let it dry at room temperature under dark conditions; and 4) The device was assembled by inserting the membrane, intermediate 1 and intermediate 2 layers inside the laminated device.

### Alternative Devices:

In an alternative embodiment of the invention, enzyme-linked complexes composed of “hormone/anti-hormone antibody-HRP conjugates” will be generated by incubating hormone and anti-hormone antibody-HRP conjugates.

In this alternative embodiment, the vertical flow microfluidic device will comprise four layers made of porous materials: an inlet layer configured for adding the resulting solution; a membrane layer comprising a nitrocellulose membrane functionalized with anti-hormone antibody where the complexes will bind and be immobilized; an absorbent/barrier layer where the solution containing anti-hormone antibody-HRP conjugates will be adsorbed; and a detection layer containing the oxidizing agent and chromogenic substrate TMB stored.

In use, the solution containing “hormone/anti-hormone antibody-HRP conjugates” will be added to the inlet layer. The complexes will be immobilized on membrane layer (the complexes will bind to the immobilized anti-hormone antibody) while the solution containing anti-hormone antibody-HRP conjugates will pass through and it will be absorbed in in the adsorbent/barrier layer. A washing buffer will be added to the membrane layer. The solution will pass through and it will be absorbed on adsorbent/barrier layer. Layer 3 will be manually removed, bringing the membrane layer and the detection layer into contact. A buffer solution will be added to the membrane layer and incubated. A blue color will be generated if complexes are retained on the membrane layer. Any color change will be quantified visually and using a smartphone software on the membrane layer or detection layer.

### REFERENCES

[1] A. Sena-Torralba, R. Alvarez-Diduk, C. Parolo, A. Piper, A. Merkoçi, Chem. Rev. 2022, 122, 14881.
[2] E. Noviana, T. Ozer, C. S. Carrell, J. S. Link, C. McMahon, I. Jang, C. S. Henry, Chem. Rev. 2021, 121, 11835.
[3] C. Carrell, J. Link, I. Jang, J. Terry, M. Scherman, Z. Call, Y. Panraksa, D. S. Dandy, B. J. Geiss, C. Henry, 2020.
[4] M. S. Verma, M. N. Tsaloglou, T. Sisley, D. Chrisfodouleas, A. Chen, J. Milette, G. M. Whitesides, Biosens. Bioelectron. 2018, 99, 77.
[5] O. Kap, V. Kilic, J. G. Hardy, N. Horzum, Analyst 2021, 146, 2784.
[6] T. T. Wang, C. K. Lio, H. Huang, R. Y. Wang, H. Zhou, P. Luo, L. S. Qing, Talanta 2020, 206, 5.
[7] C. M. Cheng, A. W. Martinez, J. L. Gong, C. R. Mace, S. T. Phillips, E. Carrilho, K. A. Mirica, G. M. Whitesides, Angew. Chem.-Int. Edit. 2010, 49, 4771.
[8] L. Fabiani, V. Mazzaracchio, D. Moscone, S. Fillo, R. De Santis, A. Monte, D. Amatore, F. Lista, F. Arduini, Biosens. Bioelectron. 2022, 200, 9.
[9] S. Kasetsirikul, M. Umer, N. Soda, K. R. Sreejith, M. J. A. Shiddiky, N. T. Nguyen, Analyst 2020, 145, 7680.
[10] R. C. Murdock, L. Shen, D. K. Griffin, N. Kelley-Loughnane, I. Papautsky, J. A. Hagen, Anal. Chem. 2013, 85, 11634.
[11] B. Pang, C. Zhao, L. Li, X. L. Song, K. Xu, J. Wang, Y. S. Liu, K. Y. Fu, H. Bao, D. D. Song, X. J. Meng, X. F. Qu, Z. P. Zhang, J. Li, Anal. Biochem. 2018, 542, 58.
[12] M. F. Abate, M. G. Ahmed, X. R. Li, C. Y. Yang, Z. Zhu, Lab Chip 2020, 20, 3625.
[13] A. Apilux, Y. Ukita, M. Chikae, O. Chailapakul, Y. Takamura, Lab Chip 2013, 13, 126.
[14] C. A. Chen, H. Yuan, C. W. Chen, Y. S. Chien, W. H. Sheng, C. F. Chen, Lab Chip 2021, 21, 1908.
[15] G. P. dos Santos, C. C. Correa, L. T. Kubota, Sens. Actuator B-Chem. 2018, 255, 2113.
[16] J. Bhardwaj, A. Sharma, J. Jang, Biosens. Bioelectron. 2019, 126, 36.

1.) A vertical flow microfluidic device configured for performing chromogenic assays, comprising:
an inlet layer for delivering a solution comprising enzyme-linked complexes,
a membrane layer comprising a porous membrane for retaining, immobilizing or adsorbing the enzyme-linked complexes,
at least one absorbent/barrier layer for absorbing the solution comprising enzyme-linked biorecognition molecule conjugates, and
a detection layer configured for comprising a chromogenic substrate and changing color when the enzyme-linked complexes react with the chromogenic substrate.

2.) The vertical flow microfluidic device according to claim 1, wherein the solution comprising enzyme-linked complexes further comprises a plurality of:
an analyte/primary (anti-analyte) enzyme-linked biorecognition molecule conjugate;
an analyte/primary (anti-analyte) biorecognition molecule/secondary (anti-primary biorecognition molecule) enzyme-linked biorecognition molecule conjugate;
an analyte immobilized on a support/primary (anti-analyte) enzyme-linked biorecognition molecule conjugate;
an analyte immobilized on a support/primary (anti-analyte) biorecognition molecule/secondary (anti-primary biorecognition molecule) enzyme-linked biorecognition molecule conjugate;
a biorecognition molecule/analyte/primary (anti-analyte) enzyme-linked biorecognition molecule conjugate;
a biorecognition molecule/analyte/primary (anti-analyte) biorecognition molecule/secondary (anti-primary biorecognition molecule) enzyme-linked biorecognition molecule conjugate;
a biorecognition molecule immobilized on a support/analyte/primary (anti-analyte) enzyme-linked biorecognition molecule conjugate; or
a biorecognition molecule immobilized on a support/analyte/primary (anti-analyte) biorecognition molecule/secondary (anti-primary biorecognition molecule) enzyme-linked biorecognition molecule conjugate.

3.) The vertical flow microfluidic device according to claim 2, wherein the solution comprising enzyme-linked complexes further comprises a buffer, chromogenic substrate, or oxidizing agent for the enzyme of the enzyme-linked biorecognition molecule conjugates.

4.) The vertical flow microfluidic device according to claim 2, wherein the analyte comprises cells, viruses, virus particles, antigens, antibodies, proteins, protein fragments, hormones, peptides, DNA, RNA, toxins, small molecules, metabolites and/or drugs.

5.) The vertical flow microfluidic device according to claim 2, wherein the enzyme of the enzyme-linked biorecognition molecule conjugate comprises HRP, ALP, or β-galactosidase.

6.) The vertical flow microfluidic device according to claim 2, wherein the biorecognition molecule comprises DNA fragments, RNA fragments, antibodies, antibodies fragments, or engineered molecules.

7.) The vertical flow microfluidic device according to claim 2, wherein the immobilization support comprises particles, magnetic beads, and other materials.

8.) The vertical flow microfluidic device according to claim 2, wherein the chromogenic substrate comprises 2,2′-Azinobis [3-ethylbenzothiazoline-6-sulfonic acid]-diammonium salt, ABTS (substrate for horseradish peroxidase, HRP), o-phenylenediamine dihydrochloride, OPD (substrate for HRP), 3,3′,5,5′-tetramethylbenzidine (substrate for HRP), p-Nitrophenyl Phosphate, PNPP (substrate for alkaline phosphatase, ALP), or o-nitrophenyl-β-D-galactopyranoside, ONPG (substrate for β-galactosidase).

9.) The vertical flow microfluidic device according to claim 3, wherein the oxidizing agent is hydrogen peroxide.

10.) The vertical flow microfluidic device according to claim 1, wherein the chromogenic substrate is configured for storage in dried and lyophilized forms.

11.) The vertical flow microfluidic device according to claim 1, wherein the membrane layer comprises a nitrocellulose membrane.

12.) The vertical flow microfluidic device according to claim 11, wherein the nitrocellulose membrane is configured for specifically or non-specifically adsorbing, retaining or immobilizing the enzyme-linked complexes.

13.) A method of using vertical flow microfluidic device according to claim 1 comprising the steps of:
Delivering a solution comprising enzyme-linked complexes onto the inlet layer
Adsorbing, retaining or immobilizing enzyme-linked complexes on the membrane layer
Absorbing the solution containing enzyme-linked biorecognition molecule conjugates in the absorbent/barrier layer
Optionally adding a washing buffer to the membrane layer
Absorbing the washing buffer in the absorbent/barrier layer
Removing the absorbent/barrier layer
Contacting the complexes in membrane layer with the detection layer comprising the chromogenic substrate and incubating with a solution comprising buffer, chromogenic substrate, or oxidizing agent to react and cause a color change
Optionally removing the membrane layer, and
Detecting the color change.

14.) The method according to claim 13, comprising the step of:
Before contacting the membrane layer with the detection layer, adding chromogenic substrates to the detection layer.

15.) The method of using vertical flow microfluidic device according to claim 13, wherein the color change is detected on the membrane layer or the detection layer.

16.) The method of using vertical flow microfluidic device according to claim 13, wherein the step of detecting the color change further comprises quantifying the color change.

17.) The method of using vertical flow microfluidic device according to claim 16, wherein quantifying the color change comprises using a smartphone for quantifying the color change.
